(19) United States
(12) Patent Application Publication
(10) Pub. No.: US 2009/0130042 A1
(43) Pub. Date: May 21, 2009

(54) Topical composition

(75) Inventors: Teanoosh Moaddel, Watertown, CT (US); Pradeep Yadav, Brooklyn, NY (US); Alexander Lips, New Canaan, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc., d/b/a Unilever

(21) Appl. No.: 11/985,796

(22) Filed: Nov. 16, 2007

(51) Int. Cl. A61K 8/06; A61Q 15/00

(52) U.S. Cl. 424/65

(57) ABSTRACT

The invention is directed to a topical composition suitable to control sweat. The composition comprises a low HLB lipid and a silicone oil whereby the composition has at least one metastable phase formed during topical application to the skin. The composition does not interfere with thermoregulation of the body, and yields a quantifiable cooling effect when applied.

### FIELD OF THE INVENTION

The present invention is directed to a topical composition that is suitable to control sweat. More particularly, the present invention is directed to a topical composition comprising a low HLB lipid and a silicone oil whereby the composition has at least one metastable amphiphile phase which is formed during topical application. When formulated with a fatty alcohol, the topical composition of this invention unexpectedly absorbs sweat and allows the same to evaporate so that additional sweat or perspiration may be absorbed by the composition. Moreover, the composition of the present invention surprisingly controls sweat without interfering with thermoregulation of the body, and yields a quantifiable cooling effect as well as antimicrobial benefits.

### BACKGROUND OF THE INVENTION

Treatment of sweat is commonly done in one of two ways. For individuals with mild cases of sweating, effective treatment may be achieved through the application of chemical antiperspirants. For individuals with a more severe case of sweating (i.e., hyperhidrosis), iontophoretic treatment may be necessary, and such treatment typically involves the electrical introduction of ions into the skin to block the sweat pore.

When treating sweat, many consumers would prefer not to use devices that involve electrical introduction of ions. Conventional topical compositions, like antiperspirants, are generally preferred, and however, such compositions are formulated to block sweat in pores. The blocking of sweat in pores is often not a preferred solution since pore blocking traps perspiration thereby interfering with thermoregulation of the body.

It is of increasing interest to develop a means for controlling sweat and cooling the body in a manner that does not utilize electrical current and that does not block secretary glands thereby preventing thermoregulation of the body. The present invention, therefore, is directed to a topical composition suitable to control sweat. The topical composition of this invention has at least one metastable amphiphile phase which is formed when the composition is being topically applied. The composition can comprise a fatty alcohol, and unexpectedly absorb sweat and allows for the evaporation of the same so that thermoregulation of the body is not prevented. Moreover, the composition of the present invention yields a quantifiable cooling effect as well as antimicrobial benefits.

### ADDITIONAL INFORMATION

Efforts have been disclosed for treating hyperhidrosis. In WO 2007/046102, sweating disorders are treated with a therapeutically effective amounts of oxybutynin, tolterodine or a substituted benzamide.

Other efforts have been disclosed for treating sweat. In U.S. Pat. No. 5,593,663, antiperspirant materials and compositions are described.

Still other efforts have been disclosed for making compositions for treating skin. In U.S. Patent Publication No. 2002/0028223 A1, anhydrous cosmetic compositions with a crosslinked siloxane elastomer gel are described.

None of the additional information above describes a composition that has at least one phase that is a metastable amphiphile phase during application whereby the composition absorbs sweat and allows for the evaporation of the same so that skin pores are not occluded and body thermoregulation is not interfered with.

### SUMMARY OF THE INVENTION

In a first aspect the present invention is directed to a composition comprising:
(a) silicone oil;
(b) lipid; and
(c) water
   wherein the composition is a flowable emulsion, the flowable emulsion comprising multiple phases and further wherein at least one metastable amphiphile phase is formed during topical application to skin.

In a second aspect, the present invention is directed to a method for cooling skin and controlling sweat by topically applying to the skin the composition described in the first aspect of this invention.

Additional aspects of the present invention will more readily become apparent from the description and examples which follow.

Metastable amphiphile phase, as used herein, is meant to mean a phase that comprises an amphiphile and that is forced, with the preferred being the forced phase over the equilibrium phase. Amphiphile is defined to mean having a polar head and a hydrophobic tail wherein the same is meant to include a lipid like glyceryl monolaurate. Microemulsion, as used herein, means an emulsion with dispersed droplets of less than about 200 nm.

Cooling effect is meant to mean reducing the temperature of skin, and preferably, from about 1 to about 2° C. upon application. The cooling effect is meant to include the effect that results from water and/or silicone evaporation, as well as crystal (e.g., formed by lipid and alcohol when used) melting within the topical composition resulting from an endothermic transition. Less viscous, as used herein is meant to mean a decrease in viscosity (ΔV) of about 5 to 10%, wherein the composition of the present invention is less viscous when silicone and/or water begin to evaporate therefrom when the composition is free of elastomer and when the composition is being applied. Skin, as used herein, is meant to mean skin on the face and body. The composition of this invention can be a base composition or an end use composition and the same may be sold in any consumable acceptable form such as an encapsulated material for use in a bar, liquid, gel, stick, roll-on formulation, cream, fabric applied formulation, mousse, lotion, ointment, cosmetic or foundation. Flowable emulsion is meant to mean an emulsion with a viscosity of less than about 175,000 cps at ambient temperature as determined with a Brookfield LV Viscometer (TC Spindle, 5 rpm, 30 sec.). Substantially free of antiperspirants (i.e., astringent salts) means less than about 4.0%, and preferably, less than about 0.02% by weight antiperspirant (e.g., aluminum chlorohydrate) in the topical composition, but most preferably, no antiperspirant in the composition. Antimicrobial effect means a bacterial reduction of at least about 0.3 log₁₀. Liquid crystal, as used herein, means a substance that exhibits a phase of matter that has properties between a conventional liquid and a solid crystal. Gel phase, as used herein, means a colloid in which the dispersed phase has combined with the dispersion medium to produce a semisolid material. Lamellar liquid crystals are the result of the stacking of bilayers. Hexagonal liquid crystals are the result of cylindrical units stacking in a hexagonally packed array, and cubic liquid crystals can be in the form of packed spherical or cylindrical units or may be bicontinuous in nature. Such liquid crystals are described in greater detail in Laughlin, The Aqueous Phase Behaviour of Surfactants, Academic Press, 1996; and Bicontinuous Liquid Crystals, Surfactant Science Series, Vol. 127, Matthew L. Lynch, Patrick T. Spicer. The term comprising, as used herein, is meant to include consisting essentially of and consisting of.

### DETAILED DESCRIPTION OF THE PREFERRED EMBODIMENTS

The only limitation with respect to the type of silicone oil that may be used in this invention is that the same can be used in a topical composition and is compatible with components that yield a metastable amphiphilic phase upon application. Illustrative non-limiting examples of the type of silicone oils that may be used in this invention include those generally classified as volatile cyclic silicones with a viscosity of less than about 10 centistokes as determined with a Ubbelohde Viscometer at ambient temperature. Suitable volatile silcones that may be used in this invention include cyclomethicones like D₅ (cyclopentasiloxane) as made available by Dow Corning, as well as D₄ and D₆ silicones (cyclobutasiloxane and cyclohexasiloxane, respectively) made commercially available by suppliers like Shin-Etsu Silicones of America, whereby such silicone oils may be used either alone or in combination with each other. The preferred silicone oil that may be used in this invention is D₅.

The amount of silicone oil used in the topical composition of this invention is typically from about 4 to about 35%, and preferably, from about 5 to about 20%, and most preferably, from about 10 to about 15% by weight, based on total weight of the composition and including all ranges subsumed therein.

As to the lipid that may be used in this invention, the same is limited only to the extent that it is suitable for use in a topical composition that yields a metastable amphiphilic phase during application. Desired lipids suitable for use in this invention often have an HLB of less than about 12, and preferably, less than about 8, and most preferably, less than about 6. Preferred lipids suitable for use in this invention include glyceryl monostearate, glyceryl monoisostearate, glyceryl monomyristate, glyceryl monoleate, diglyceryl monoisostearate, propylene of glycol monostearate, propylene glycol monoisostearate, propylene glycol monocaprylate, sorbitan monoisostearate, sorbitan monocaprylate, sorbitan monoisooleate, glyceryl monolaurate, glyceryl monocaprylate, glyceryl monocaprate, mixtures thereof or the like. In a preferred embodiment, the lipid used in this invention is glyceryl monolaurate, made available by suppliers like Fitz Chem Corporation under the name Monomuls® 90-L12.

Typically, the lipid makes up from about 4 to about 35%, and preferably, from about 5 to about 20%, and most preferably, from about 10 to about 15% by weight of the composition, based on total weight of the composition and including all ranges subsumed therein.

In a preferred embodiment, the topical composition of the present invention further comprises an alcohol like a fatty alcohol and/or an ester like a fatty ester. Illustrative non-limiting examples of alcohols which may be used include behenyl alcohol, isopropyl myristate, caprylic alcohol, cetearyl alcohol, coconut alcohol, decyl alcohol, isocetyl alcohol, lauryl alcohol, oleyl alcohol, palm kernel alcohol, isostearyl alcohol, stearyl alcohol, cetyl alcohol, tallow alcohol, tridecyl alcohol, myristyl alcohol, mixtures thereof, or the like. In a preferred embodiment, however, the alcohol used comprises isostearyl alcohol. In a most preferred embodiment, the alcohol is a C₁-C₆ alkyl branched isostearyl alcohol (e.g., methyl branched) made available by suppliers like Uniqema under the name Prisorine 3515.

Illustrative fatty esters include isopropyl myristate, isopropyl stearate, isopropyl oleate, isosonyl stearate or mixtures thereof.

Typically, the amount of alcohol and/or ester used in the topical composition of this invention is from about 1 to about 15, and preferably, from about 2 to about 10, and most preferably from about 2.5 to about 5% by weight, based on total weight of the topical composition and including all ranges subsumed therein.

In an especially preferred embodiment, the weight ratio of lipid to alcohol and/or ester is from about 6:1 to about 1:6, and preferably, about 5:1 to about 1:5, and most preferably, from about 4:1 to about 1:4 where the weight ratio is based on total weight of lipid and alcohol in the topical composition and including all ratios subsumed therein.

In yet another preferred embodiment, and especially when a silky and less draggy sensation is desired, elastomer may be used in the topical compositions of this invention. When used, the elastomer typically causes the composition to transform during application from an opaque cream-like material to a gel.

The elastomer which may be used is typically one which may be delivered (or carried) in the silicone oils described herein. Such elastomers are often classified as non-emulsifying elastomers having an average number (Mn) molecular weight in excess of 2,000, preferably, in excess of 5,000, and most preferably, in the range from about 10,000 to about 20 million, including all ranges subsumed therein.

Often, the elastomers are formed from a divinyl compound which has at least two free vinyl groups, reacting with Si—H linkages of a polysiloxane backbone. Elastomer and oil compositions are commercially available with Cyclomethicone and Vinyl Dimethicone Methicone Cross Polymer, delivered as 20-35% elastomer in a cyclomethicone carrier. A related elastomer and oil composition with Crosslinked Stearyl Methyl Dimethyl Siloxane Copolymer is available as Gransil SR-CYC (25-35% elastomer in a cyclomethicone carrier) from Grant Industries, Inc., Elmwood Park, N.J. The commercial products from, for example, Grant Industries ordinarily are further processed by subjecting them to a high pressure (approximately 5,000 psi) treatment in a Sonolator with recycling in 10 to 60 passes. Sonolation achieves a resultant fluid with elastomer average particle size ranging from 0.2 to 10 micron, preferably 0.5 to 5 micron. Viscosity is preferred often when ranging between 300 and 20,000 cps at 25° C., as measured by a Brookfield LV Viscometer (size 4 bar, 60 rpm, 15 sec). In an especially preferred embodiment, a most desired non-emulsifying elastomer is a cyclomethicone/dimethicone cross-polymer made commercially available in silicon oil by suppliers like Dow Chemical under the name DC9040 and DC9045, and Shin-Etsu under the names KSG-14 and KSG-15 elastomer gels. Typically, elastomers can make up to about 40% by weight of the total weight of the elastomer and oil composition. The preferred elastomer and silicon oil mixture is KSG-15 which has about 5-12% by weight cyclomethicone/vinyl dimethicone cross-polymer in a cyclomethicone carrier/oil.

Typically, however the amount of elastomer (not including carrier oil), used in topical composition of this invention (when elastomer is desired) is from about 0.05 to about 12%, and preferably, from about 0.1 to about 8%, and most preferably, from about 0.5 to about 6% by weight, based on total weight of the topical composition and including all ranges subsumed therein.

Water makes up the balance of the topical composition of this invention and often makes up at least about 50%, and preferably, at least about 60%, and most preferably, from about 62 to about 80% by weight of the topical composition, including all ranges subsumed therein.

As previously mentioned, the topical compositions described herein may be base compositions or end use compositions. When base (i.e., carrier) compositions, optional additives may be used.

Viscosity builders, for example can be utilized as an optional portion of the topical compositions according to the present invention. Viscosity builders include silicone gums, crosslinked acrylates (e.g. Carbopol 982®), hydrophobically-modified acrylates (e.g. Carbopol 1382®), polyacrylamides (e.g. Sepigel 305®), acryloylmethylpropane sulfonic acid/salt polymers and copolymers (e.g. Aristoflex HMB® and AVC®), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pecin and combinations of these gums. Amounts of the viscosity builder may range from 0.0001 to 15%, usually from 0.001 to 10%, optimally from 0.01 to 5% by weight of the topical composition, including all ranges subsumed therein.

Adjunct humectants may be employed in the present invention if desired. These are generally polyhydric alcohol-type materials. Typical polyhydric alcohols include glycerol, propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of adjunct humectant may range anywhere from 0.5 to 50%, preferably between 1 and 15% by weight of the composition.

Surfactants, if desired, may also be present in topical compositions of the present invention. Total concentration of the surfactant when present may range from about 0.1 to about 35%, preferably from about 1 to about 25%, optimally from about 1 to about 20% by weight of the topical composition, and being highly dependent upon the type of end use product. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di- C₈-C₂₀ fatty acids; and polyoxyethylene sorbitan as well as combinations thereof. Alkyl polyglycosides and saccharide fatty amides (e.g. methyl gluconamides) and trialkylamine oxides are also suitable nonionic surfactants.

Preferred anionic surfactants include soap, alkyl ether sulfates and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, C₈-C₂₀ sarcosinates, C₈-C₂₀ acyl lactylates, sulfoacetates and combinations thereof. An often most preferred anionic surfactant is sodium dodecyl sulfate (SDS).

Useful amphoteric surfactants include cocoamidopropyl betaine, C₁₂-C₂₀ trialkyl betaines, sodium lauroamphoacetate, and sodium laurodiamphoacetate.

Sunscreen agents may also be included in topical compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide and zinc oxide. Amounts of the sunscreen agents when present may generally range from 0.1 to 30%, preferably from 2 to 20%, optimally from 4 to 10% by weight of the topical composition.

Preservatives can desirably be incorporated into the topical compositions of this invention to protect against the growth of potentially harmful microorganisms. Particularly preferred preservatives are phenoxyethanol, methyl paraben, propyl paraben, imidazolidinyl urea, dimethyloldimethylhydantoin, ethylenediaminetetraacetic acid salts (EDTA), sodium dehydroacetate, methylchloroisothiazolinone, methylisothiazolinone, iodopropynbutylcarbamate and benzyl alcohol. The preservatives should be selected having regard for the use of the composition and possible incompatibilities between the preservatives and other ingredients. Preservatives, when desired, are preferably employed in amounts ranging from 0.01% to 2% by weight of the composition.

Topical compositions of the present invention may optionally include vitamins. Illustrative vitamins are Vitamin A (retinol), Vitamin B₂, Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present in compositions according to the present invention may range from 0.001 to 10%, preferably from 0.01% to 1%, optimally from 0.1 to 0.5% by weight of the topical composition.

Another type of useful optional substance can be that of an enzyme such as amylases, oxidases, proteases, lipases or combinations. Particularly preferred is superoxide dismutase, commercially available as Biocell SOD from the Brooks Company, USA.

Skin lightening compounds may be included in the topical compositions of the invention. Illustrative substances are placental extract, lactic acid, niacinamide (Vitamin B₃), arbutin, kojic acid, ferulic acid, resorcinol and derivatives including 4-substituted resorcinols and combinations thereof. Amounts of these agents (when desired) niay range from about 0.1 to about 10%, preferably from about 0.5 to about 6% by weight of the topical composition.

Omega fatty acids may also be employed in the topical composition of the present invention. Such acids include linoleic acid, oleic acid, petroselinic acid, linolenic acid, linoleaidic acid, elaidic acid, myristoleic acid, mixtures thereof, or the like. In a preferred embodiment, the omega fatty acids employed include conjugated linoleic acid and petroselinic acid. In a most preferred embodiment, the omega fatty acid employed is conjugated linoleic acid.

When used, omega fatty acid typically makes up from 0.01 to about 15, and preferably, from about 0.5 to about 10, and most preferably, from about 1 to about 7% by weight of the topical composition, based on total weight of the topical composition and including all ranges subsumed therein.

Desquamation promoters may be present. Illustrative are the alpha-hydroxycarboxylic acids and beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic, lactic and malic acids. Salicylic acid is representative of the beta-hydroxycarboxylic acids. Amounts of these materials when present may range from about 0.01 to about 15% by weight of the topical composition.

A variety of herbal extracts may optionally be included in compositions of this invention. The extracts may either be water soluble or water-insoluble carried in a solvent which respectively is hydrophilic or hydrophobic. Water and ethanol are the preferred extract solvents. Illustrative extracts include those from green tea, chamomile, licorice, aloe vera, grape seed, citrus unshui, willow bark, sage, thyme and rosemary.

Also included may be such materials as lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Amounts of these materials (when their benefits are desired) may range from about 0.000001 to about 10%, preferably from about 0.0001 to about 1% by weight of the topical composition.

Colorants, opacifiers and abrasives may also be included in compositions of the present invention. Each of these substances, if desired, may range from about 0.05 to about 5%, preferably between 0.1 and 3% by weight of the composition.

Oil control additives are often a preferred optional additive that may be employed in the topical compositions of this invention. Such oil control additives include spheroids like silica modified ethylene/methacrylate copolymer microspheres, talc modified ethylene/methacrylate copolymer microspheres, mixtures thereof or the like.

Other examples of the types of spheroids suitable for use in this invention include those comprising polyolefins like polyethylene, polypropylene and/or polybutylene-based polymers, polyamides (like nylon fibers), mixtures thereof or the like. Still other spheroids suitable for use in this invention include those comprising polyurethane, polystyrene, epoxy resins, urea resins, silicone resins, mixtures thereof or the like.

In a preferred embodiment, the spheroids used in this invention comprise polyethylenes, or are talc comprising particles or mixtures thereof. The former are often sold under the names Cerapure (made commercially available by Shamrock), Asensa (made commercially available by Honeywell) and Miperon (made commercially available by Mitsui Petrochemical Industries, Ltd.). Another preferred polyethylene-based spheroid is sold under the name CL-2080 (made commercially available by Kobo Industries). Other preferred spheroids suitable for use in this invention include nylons (e.g., nylon-12) sold under the name SP-10 which is made commercially available by Kobo Industries. Still other preferred spheroids suitable for use in this invention include those comprising copolymers of ethylene and methacrylate that contain silica or talc and sold under the names SPCAT-I2 and DSPCS-I2, respectively, both of which are also made commercially available by Kobo Industries. Other spheroids comprising polystyrenes and polymethyl methacrylate (sold, for example, under the names Ganzpearl GS-0605 and GME0380, respectively) and made available from Presperse are also often preferred.

Even other spheroids suitable for use in this invention include natural polymeric spheroids like those which comprise starch and those which comprise silk, the former, for example, made available from National Starch and Chemical and the latter, for example, made available by Engelhard Corporation. Still other natural polymeric spheroids suitable for use in this invention include those natural polymeric spheroids comprising cellulose such as Celluflow and Cellulo Beads, the former made commercially available by Chisso Corporation and the latter made available by Kobo Industries.

When selecting spheroids for use in this invention, typically those often desired have an oil absorption number from about 0.2 to about 15 g/g, and preferably, from about 0.2 to about 12 g/g, and most preferably, from about 0.9 to about 8 g/g, including all ranges subsumed therein, where g/g means gram of sebum absorbed per gram of spheroid at ambient temperature.

In a more preferred embodiment of this invention, the spheroids employed make up from about 1 to about 15, and most preferably, from about 5 to about 10% by weight of the topical composition, based on total weight of the topical composition and including all ranges subsumed therein. In another more preferred embodiment, the spheroids have an approximate diameter from about 4 to about 40, and most preferably, from about 5 to about 35 microns, including all ranges subsumed therein. Optionally, but often preferably, the spheroids, when employed in this invention, are used with thickening agents (i.e., inorganics) that are suitable to thicken oil (i.e. sebum) at the temperature of the skin that the skin care composition is applied to. Illustrative but non-limiting examples of the types of thickening agents suitable for use in this invention include bismuth oxychloride, mica, fumed silica, micronized teflon, aluminum silicate, magnesium aluminum silicate, bentonite, calcium silicate, chalk, clay (like kaolin and laponite), hydrated silica zinc oxide, silica, talc, mica, titanium dioxide, magnesium oxide, alumina, calcium carbonate, mixtures thereof or the like. In a preferred embodiment, the thickening agent, when employed, has an approximate diameter from about 0.5 to about 3.5, more preferably, from about 0.7 to about 2.5, and most preferably, from about 0.8 to about 1 micron, including all ranges subsumed therein. Typically, the thickening agent makes up from about 0.001 to about 10% by weight of the topical composition, based on total weight of the topical composition and including all ranges subsumed therein.

When making the topical composition of the present invention, the desired ingredients are combined in no particular order and heated to at least about 50° C. at atmospheric pressure and while stirring. Stirring should continue until a homogeneous cream is made. Stirring is typically stopped when the desired topical composition reaches about ambient temperature in the absence of heating.

In a preferred embodiment, the topical composition of the present invention consists essentially of silicone oil, alcohol, lipid and water. In another preferred embodiment, the topical composition of the present invention consists essentially of silicone oil, lipid, oil control additive, water and elastomer.

When utilizing the compositions of the present invention, the consumer is directed to apply the composition to the skin and to leave the composition on the skin to realize all benefits. In a preferred embodiment, the consumer is directed to use from about 0.75 to about 3.5 mg of topical composition for about every 2 cm² of skin, including all ranges subsumed therein. In a most preferred embodiment, the consumer is directed to use from about 1.5 to about 2.5 mg of topical composition for about every 2 cm² of skin.

The topical composition of the present invention, when applied, is suitable to reduce the temperature of the skin as described herein. Without elastomer, the composition is typically an opaque cream and becomes a liquid upon application.

Moreover, the topical composition of this invention absorbs water and sebum (i.e., sweat) and the absorbed sweat has, unexpectedly; a water activity of at least about 0.7, and preferably, at least about 0.8, and most preferably, at least about 0.9 to about 0.99 within the topical composition.

The absorbed sweat within the topical composition of this invention unexpectedly evaporates or circulates back into the environment and does not occlude or block pores thereby keeping the film of composition on the consumer active (e.g., suitable to absorb more sweat). Such a film does not interfere with thermoregulation since it remains active. Furthermore, the film of composition on the skin of the consumer has an antimicrobial effect notwithstanding the fact that the film cools the skin and remains an active film suitable to absorb additional sweat.

The topical compositions of the present invention can be incorporated into an insoluble substrate for application to the skin such as in the form of a treated wipe.

A wide variety of packaging can be employed to store and deliver the topical compositions described herein. Packaging is often dependent upon the type of personal care end-use. For instance, leave-on skin lotions and creams generally employ plastic containers with an opening at a dispensing end covered by a closure. Typical closures are screw-caps, non-aerosol pumps and flip-top hinged lids. Packaging can include a roll-on ball on a dispensing end. Alternatively, these types of topical compositions may be delivered in a stick composition formulation in a container with propel-repel mechanism where the stick moves on a platform towards a dispensing orifice. All of the aforementioned are considered potential packaging within context of the present invention.

The Examples are provided to facilitate an understanding of the present invention and they are not meant to limit the scope of the claims.

### EXAMPLE 1

Topical compositions (base) of the present invention were made by blending the following components at about 50° C. The compositions were allowed to cool with mixing to about ambient temperature prior to use.

| Component | Weight % |
|---|---|
| Glyceryl monolaurate | 10-15 |
| Elastomer (8%) &silicone oil (KSG-15) | 10-15 |
| Isostearyl alcohol | 2-5 |
| Water | Balance |

Artificial sweat was prepared by mixing about 0.56 g KCL; about 0.118 g NaCl; about 0.021 aluminum chloride; about 0.089 L(+) lactic acid; about 0.054 L-methionine; about 0.005 mucic acid; about 0.018 urea and water to balance.

The resulting homogenous topical composition (which was opaque and cream-like) was applied onto a VWR microslide (1.5 in×1 in, 1 mm) using a 25 mm film applicator (Sheen Instruments) having a 37 micron gap size.

The glass slide with the 37 micron thick film of composition was mounted onto a Leitz Laborlux 12 Pol S optical microscope fitted with cross-polarizers and a Linkam heating stage set at about 35° C.

The product film was allowed to dry at which point visible crystals and oil were observed. One drop of artificial sweat was applied to the dry film of composition. It was immediately observed that the crystals in the dry product film readily converted to liquid crystals (e.g., mixture of cubic and hexagonal liquid crystal) upon contact of film with artificial sweat. This conversion of crystal to liquid crystal upon contact with sweat illustrates the water binding capacity of the topical composition of this invention.

Sweat/water in the film was subsequently allowed to evaporate from the product film which demonstrated the regeneration of the crystal as observed through the optical microscope fitted with cross-polarizers. Another drop of artificial sweat was then re-applied after which the conversion of the film back to a liquid crystal was observed. The results indicate that the topical composition made according to this invention remains active and is suitable to continuously absorb water after water evaporates from the same.

A group of skilled panelists applied the topical compositions made in this example to evaluate the cooling perception of the composition. About 70% of the panelists perceived a cooling perception on application. This perceived cooling effect was then confirmed by monitoring the skin surface temperature using a thermal camera (made available by Fluke® Thermal Cameras), after application of about 2.5 mg/cm² of topical composition to the forearm of the panelists. Observed was a reduction in temperature of about 2° C.

### EXAMPLE 2

A topical composition (base) and a control composition were made in a manner consistent with the procedure described in Example 1. Composition (I) was made consistent with this invention and comprises glycerol monolaurate. Composition (II) is the control and employs surfactant in lieu of glycerol monolaurate.

### I. Topical Composition (Base)

| Component | wt % | g |
|---|---|---|
| Water | 69.75 | 20.925 |
| Glycerol monolaurate | 13 | 3.9 |
| Isostearyl alcohol | 3.25 | 0.975 |
| KSG-15 silicone elastomer | 14 | 4.2 |
| (8%) | | |

### II. Topical Composition (Control)

| Component | wt % | g |
|---|---|---|
| Water | 78.37 | 20.925 |
| Isostearyl alcohol | 3.65 | 0.975 |
| KSG-15 silicone elastomer | 15.73 | 4.2 |
| (8%) | | |
| Octyl phenolethoxylate | 2.25 | 0.6 |
| Surfactant (Dow) | | |

Commercially available silicone sheets were cut into 4×4 cm sections and placed on Tryptic Soy Agar (TSA) plates. The composition made according to this invention and the control (2 mg/cm², total of 32 mg) were applied to separate silicone sheets and rubbed over the surface with a gloved finger. An untreated section served as an organism control. The treated and untreated sections were incubated for 10 minutes (30° C.), after which 0.1 ml of test organism was applied to the silicone and spread over the surface. The sections were incubated for 30 more minutes at 30° C. After incubation, the sections were carefully placed into 10 ml of Letheen broth (comprising neutralizers) and vortexed on high for 1 minute. This broth was serially diluted 10-fold in Letheen broth, and 1 ml aliquots were spread-plated across the surface of 3 Letheen agar plates. The plates were incubated for ˜27 hours at 32° C. and counted using a Quebec colony counter. Staphylococcus aureus was the bacteria used. The results indicated that the composition made according to this invention had a bacteria reduction of at least 0.3 log₁₀ greater than the control, in addition to the fact that the composition is one which results in a film that cools and is suitable to control sweat without interfering with thermoregulation of the body.

What is claimed is:

1. A composition comprising:
(d) silicone oil;
(e) lipid; and
(f) water
wherein the composition is a flowable emulsion, the flowable emulsion comprising multiple phases and further wherein at least one metastable amphiphile phase is formed during topical application to skin.

2. The composition according to claim 1 wherein the metastable amphiphile phase is a microemulsion, a liquid crystal, a gel phase, or a mixture thereof.

3. The composition according to claim 2 wherein the liquid crystal comprises a lamellar liquid crystal, a hexagonal liquid crystal and/or a cubic liquid crystal and the gel phase comprises a lamellar gel phase.

4. The composition according to claim 1 wherein the composition reduces the temperature of skin during application.

5. The composition according to claim 4 wherein the reduction of skin temperature is from about 1 to about 2° C. during application.

6. The composition according to claim 1 wherein the composition further comprises a fatty alcohol, fatty ester or both.

7. The composition according to claim 6 wherein the fatty alcohol comprises isostearyl alcohol and the fatty ester comprises isopropyl myristate.

8. The composition according to claim 1 wherein the composition is substantially free of antiperspirants.

9. The composition according to claim 1 wherein the composition after application is not water soluble and becomes less viscous upon applying.

10. The composition according to claim 1 where in the composition is opaque prior to applying to the skin and a gel or liquid during application.

11. The composition according to claim 1 wherein the lipid has an HLB of less than about 12.

12. The composition according to claim 11 wherein the lipid is glyceryl monolaurate.

13. The composition according to claim 6 wherein water and/or silicone oil evaporates from the composition after application to form crystals suitable to absorb sweat and sebum, the crystals comprising lipid, and fatty ester and/or fatty alcohol wherein the crystals under go an endothermic transition to melt thereby cooling skin.

14. The composition according to claim 1 wherein the silicone oil is a cyclomethicone.

15. The composition according to claim 1 wherein the composition further comprises oil control additives.

16. A method for cooling skin and controlling sweat on skin comprising the steps of:
(a) applying to skin a composition comprising:
(i) silicone oil;
(ii) lipid; and
(iii) water; and
(b) allowing the composition to dry on the skin
wherein the composition is a flowable emulsion, the flowable emulsion comprising multiple phases and further wherein at least one metastable amphiphile phase is formed during application to skin.

17. The method according to claim 16 wherein the metastable amphiphile phase is a microemulsion, a liquid crystal, a gel phase or a mixture thereof.

18. The method according to claim 16 wherein the composition further comprises from about 3 to about 25% by weight silicone oil, from about 3 to about 25% by weight lipid, and a fatty alcohol, fatty ester or both.

19. The method according to claim 18 wherein the fatty alcohol is isostearyl alcohol, the fatty ester is isopropyl myristate and the fatty alcohol and/or fatty ester and lipid are present in the composition at a weight ratio from about 1:6 to about 6:1.

20. The method according to claim 16 wherein skin cools from about 1 to about 2° C. while applying the composition.

21. The method according to claim 18 wherein the composition after application is not water soluble wherein water evaporates from the composition after application to form crystals suitable to absorb sweat and sebum.
